(19) United States
(12) Patent Application Publication
(10) Pub. No.: US 2024/0351972 A1
(43) Pub. Date: Oct. 24, 2024

(54) PREPARATION OF A CATALYST FOR THE OXIDATIVE ESTERIFICATION OF METHACROLEIN TO METHYL METHACRYLATE, FOR EXTENDING SERVICE LIFE

(71) Applicant: Röhm GmbH, Darmstadt (DE)

(72) Inventors: Andreas Rühling, Darmstadt (DE); Steffen Krill, Muehltal (DE); Florian Zschunke, Frankfurt (DE); Belaid Ait Aissa, Darmstadt (DE); Andreas Tepperis, Bad Koenig (DE); Mounir Stitou, Wuppertal (DE)

(73) Assignee: Röhm GmbH, Darmstadt (DE)

(21) Appl. No.: 18/685,379

(22) PCT Filed: Aug. 19, 2022
(86) PCT No.: PCT/EP2022/073183

§ 371 Date: Feb. 21, 2024

(30) Foreign Application Priority Data
Aug. 23, 2021 (EP) 21192495.6

(51) Int. Cl. C07C 67/44; B01J 23/42; B01J 23/44; B01J 23/52; B01J 35/45; B01J 38/08; B01J 38/64

(52) U.S. Cl. CPC C07C 67/44; B01J 23/42; B01J 23/44; B01J 23/52; B01J 35/45; B01J 38/08; B01J 38/64

(57) ABSTRACT

A process for performing a heterogeneously catalysed reaction for the oxidative esterification of aldehydes to carboxylic esters can be performed. Against this background, the process has made it possible to keep the heterogeneous, precious metal catalyst used in this method active during operation in a particularly effective manner, in order to lengthen the period between downtimes and to effect particularly sustainable catalyst management. This gives rise to the possibility of performing such processes in a very simple, economically viable and environmentally benign manner.

### FIELD OF THE INVENTION

The present invention relates to a novel process for performing a heterogeneously catalysed reaction for the oxidative esterification of aldehydes to carboxylic esters. Against this background, the present process according to the invention has made it possible to keep the heterogeneous, precious metal catalyst used in this process active during operation in a particularly effective manner, in order to lengthen the period between downtimes and to effect particularly sustainable catalyst management. This gives rise to the possibility of performing such processes in a very simple, economically viable and environmentally benign manner

### PRIOR ART

The catalytic oxidative esterification of aldehydes for preparation of carboxylic esters is described in numerous patents and references. For example, it is possible to prepare methyl methacrylate very efficiently from methacrolein and methanol.

When the readily polymerizable reactants and/or products are used or prepared, it is particularly important, for an economically viable process, to suppress polymerization as far as possible in order to achieve the high activities, selectivities and catalyst on-stream times. Particularly in the case of the costly precious metal catalysts based, for example, on Au, Pd, Ru or Rh, the catalyst on-stream time plays a crucial role. In the case of the oxidative esterification of methacrolein (MAL) to methyl methacrylate (MMA), it is moreover desirable for it to be possible to carry out the reaction in the presence of relatively high concentrations of MAL, as a result of which a higher space-time yield becomes possible, and the amount of reactants to be recycled by distillation is reduced. This has a positive effect on the process, in terms of both energy and apparatuses.

The prior art to date has not sufficiently described how a high catalyst activity, selectivity and long on-stream time can be achieved without deactivation, particularly at high MAL concentrations in the reaction mixture, or how the process can be carried out in a largely stable, disruption-free and continuous manner.

The process for the direct oxidative esterification of methacrolein to MMA has been described many times before. For example, U.S. Pat. No. 5,969,178 describes a Pd-Pb-catalysed conversion of MAL to MMA with a selectivity of 86.4% at a space-time yield (STY) of 5.5 mol of MMA/kg cat.*h. The possible MAL and methanol concentrations in the feed at the reactor inlet are discussed in detail here, but no information is given as to the composition in the reactor. The oxygen concentration in the reactor offgas is described and discussed with the following background: for instance, the oxygen concentration in the offgas, owing to the explosion limit, is to be less than 8% by volume. In addition, a lower oxygen concentration in the reactor, and also in the offgas, is said to be disadvantageous for the reaction rate. Thus, excessively low oxygen concentrations led to increased formation of by-products.

On the other hand, however, it is also pointed out that the greater the oxygen concentration, the more Pb salts have to be fed continuously to the reactor in order that the catalyst performance remains constant and good.

The use range preferred for all these reasons for a Pd-Pb catalyst is thus between the partial oxygen pressure in the offgas of 0.01 and 0.8 kg/cm² with total pressure between 0.5 and 20 kg/cm². In the best embodiment of U.S. Pat. No. 5,969, 178 of exemplary embodiment 1, the reaction is conducted at total pressure 3.0 kg/cm² and partial O₂ pressure 0.095 kg/cm² in the offgas (corresponding to 3.2 vol % oxygen in the offgas).

U.S. Pat. No. 8,450,235 disclosed the use of an NiO/Au-based catalyst at total pressure 0.5 MPa and 4% by volume of oxygen in the offgas. The selectivity for MMA was 97.2%; the space-time yield was 9.57 mol of MMA/kg cat.*h. The molar ratio of methanol to methacrolein in the feed was 4.36 (mol/mol). The calculated corresponding ratio in the reactor was 14.7 (mol/mol).

If a distillative separation of methanol and methacrolein is to take place after the oxidative esterification, as described, for example, in U.S. Pat. No. 5,969,178, it is energetically more advantageous to reduce the molar ratio of methanol to methacrolein in the reactor to less than 10 (mol/mol). In principle, it is advantageous to separate methanol from the target product, MMA, as a low-boiling azeotrope of methanol and methacrolein. If a mode of operation is selected with a low methanol to methacrolein (MAL) ratio in the reactor, less MMA is recycled together with MAL, since MMA and methanol also form a low-boiling azeotrope. The methanol-MAL azeotrope, according to U.S. Pat. No. 5,969,178, has a boiling point of 58° C. and a composition of methanol to MAL of 72.2% by weight to 27.7% by weight. The molar ratio of methanol to MAL here is 5.7. On the other hand, it should be taken into consideration that the MMA selectivity is positively influenced by the methanol excess in the reactor. In principle, the higher the methanol excess and the lower the stationary water concentration in the reactor, the higher the achievable MMA selectivity and the lower the production of methacrylic acid as one of the by-products in the process.

However, all these processes share the common feature that catalyst activity falls as operating periods get longer. Catalyst deactivation is a known phenomenon in all catalytic processes, and can be broken down into different classes. A review of catalyst deactivation is provided by Argyle et. al. in “Heterogeneous Catalyst Deactivation and Regeneration: a Review”, in Catalysts 2015, 5, 145-269.

In the case of the catalytic reaction described here, there are a plurality of parallel mechanisms that lead to a decline in catalytic activity. The catalyst is mechanically stressed and forms fragmented particles and fine particles which can no longer be retained in the reactor by the filtration system. In addition, very small constituents of the mixed oxide support, and ultimately also of the active nanoparticulate precious metal, become physically detached from the catalyst matrix or are mechanically abraded therefrom. In addition, use is often made of layered catalysts, as described for example in U.S. Pat. No. 8,450,235, leading to the possibility of active components being removed from the catalyst surface because of mechanical stress, as a result of which catalyst activity falls further.

In practice, support constituents and precious metals are found in the product effluent in the ppb or ppm range. Nevertheless, over several thousands of hours of operating time, this leads to measurable activity loss. A further activity-reducing factor is the sintering of the active precious metal species, forming larger agglomerates which have reduced activity or which no longer have any activity because, for example, the catalytic activation of oxygen or the cleavage of molecular oxygen to elemental oxygen is inhibited or even prevented. Furthermore, it should be noted that an additional reduction in selectivity can be observed when very fine catalyst particles are formed.

A further cause of catalyst deactivation is the accumulation of organic compounds from the reaction solution at the surface and in the pores of the catalyst. This process is known to those skilled in the art as fouling. Fouling as a deactivation mechanism becomes more significant in continuous process with high catalyst run times or on-stream times, because the formation and presence of absorptive and adsorptive components increase over the operating time specifically in relation to the amount of catalyst. Such absorptive and adsorptive compounds are commonly unsaturated in nature, for example (meth) acrolein and (meth) acrylic acid and the sodium salts thereof, and are converted into oligomeric or polymeric constituents over the course of continuous operation. Depending on how the reaction is performed, coking of the catalyst may also occur (see Wolf et. al. “Catalysts Deactivation by Coking” in Catalysis Reviews: Science and Engineering, 1982, 24, 329-371, which is a review article).

The formation and precipitation of such oligomers, polymers and salts from the reaction solution is also described in JP20004-345975A, precipitation only being described at those locations in the reactor where gas is introduced. An influence on the catalyst is not mentioned.

The accumulation of oligomers, polymers and salts from the catalyst causes blockage of the active centres of the catalyst, as a result of which catalyst activity falls.

The deactivation of the catalyst by adsorption of organic substances is also described by Zhang et. al. in Applied Catalysis B: Environmental (2013), 142, 329-336, where the catalyst used for the direct oxidative esterification is a Pd-Pb system. In the context of this document, batchwise washing with methanol and a hydrazine solution is used for regeneration, for which the catalyst is previously filtered in ambient air. The document does not discuss the handling of waste containing hydrazine and methacrolein or the associated safety requirements. Moreover, hydrazine is known to those skilled in the art as a reducing agent by means of which gold catalysts can reduce unsaturated compounds A gold or palladium catalyst treated with hydrazine would therefore at least partially reduce the above-described MMA to the saturated compound methyl isobutyrate, which can only be separated from the MMA by distillation expensively and with great difficulty. With regard to the typical purity of MMA for application as a monomer, the use of hydrazine should therefore be seen in a negative light and is associated with considerable additional expenditure.

Moreover, in this regard, it should be noted that hydrazine—like many amines—behaves as a base, and as a result the catalyst or the support material thereof can also become basic. Those skilled in the art know that the reaction rate of direct oxidative esterifications can be increased through elevated pH, for example in the range between 7.5 and 10; however, depending on the product, the selectivity can fall. Using the experimental description of Zhang et al., it is assumed that this influence of the pH, brought about by the hydrazine treatment, triggers a temporary increase in conversion that conceals the partial deactivation by adsorption of organic substances However, in continuous operation, the disadvantages in terms of increasing selectivities for by-products and the non-dissolution of the substances adsorbed on the catalyst surface predominate.

In addition, the regenerative effect of washing is only demonstrated for batch processes, the reason given being that the presence of methanol in continuous operation prevents fouling. However, the continuous example shown in the document only covers a time frame of less than 24 hours, which does not enable any comprehensive assessment to be made for industrially relevant catalyst on-stream times.

Consequently, the catalysts consumed or deactivated by fouling and other parallel deactivation processes have to be removed from the process, in order to be either regenerated or worked up. Direct oxidative esterification involves an oxidation catalyst and an exothermic reaction. This means that, upon exposure to ambient air and in the presence of the process-related organics, the catalyst can catalyse an exothermic reaction which may present a risk to humans, the environment, and process safety. JP 4115719B describes this very same process risk, without discussing how the catalyst can be removed from the reaction in continuous operation or how it can subsequently have organic components removed therefrom. The extent of the risk is mentioned in the document by stating that if the organic components are not removed, the catalyst heats up in air, for example during removal from the reactor, to the extent that the material burns. Accordingly, therefore, there is a risk of (self-) ignition or explosion.

Above all, the great risk to humans and the environment originating from unsaturated aldehydes such as methacrolein and acrolein must clearly be overcome. While the removal of organic adsorbates such as methacrolein or methacrylic acid, or the sodium salts thereof, from the catalyst surface is still readily achievable by washing multiple times, the removal of said organics from within the pore structure of the catalyst is considerably more difficult. In particular, methacrolein adsorbed within the pores can leave the pores extremely slowly by diffusion-based processes. This constitutes a particular process risk or safety precaution for catalyst workup, because a methacrolein-containing atmosphere may be released into the environment when the catalyst container is opened.

In addition, the slow release of, for example, methacrolein from the pores of the catalyst means that, in a short wash, the catalyst can only be partially regenerated at the surface. When the typically high porosity of heterogeneous powder catalysts is considered, it is clear for those skilled in the art that any catalyst activity recovered will only be short-lived.

Along with the catalyst loss by various mechanisms for reducing catalyst activity that occur one after the other, in particular in the liquid-phase oxidation of methacrolein to MMA, there is a significant effect on continuous plant operation and on the way in which the process and plant parts can be designed and dimensioned. Plants are in principle dimensioned on the basis of plant conditions determined under a steady state, such as the targeted conversion and the stoichiometry of the feedstocks, which are particularly relevant here. In the liquid-phase oxidation of methacrolein, the plant and the separation effect of the columns are set to a partial conversion of methacrolein in a single pass through the reaction, especially for this oxidative esterification, in a conversion range between 55% to 85% relative to the methacrolein supplied to the reactor. The conversion figure relates to the total conversion, regardless of whether there is one reactor or a plurality of reactors in a sequential configuration. If a loss of catalyst activity occurs, or a change in the space-time yield caused thereby, the composition of all product mixtures changes such that more unconverted reactants have to be recycled, until the designed plant is no longer able to achieve the nominal capacity. Moreover, this may lead to separating principles, such as the separation of azeotropes and the prevention of two-phase mixtures in separation steps not designed for this purpose, no longer working. Therefore, particularly in direct oxidative liquid-phase oxidation of methacrolein, it is an essential requirement for catalyst and process to ensure that declines in catalyst activity are kept as low as possible and to counteract the natural ageing and reduction in catalyst activity, such that the workup and space-time yield of the continuous plant meet their design criteria. The prior art set out above does not offer any satisfactory technical solution that ensures largely stable activity and space-time yields of the catalyst and reaction system.

### Problems

In light of the prior art, there was therefore a great deal of interest in improving the process of a continuously operated oxidative esterification so as to enable better and more operationally reliable catalyst management and to prolong the periods between maintenance downtimes, to remove the catalyst from the reaction at the end of its operational time or lifetime, and to remove the process-related organics safely therefrom.

A particular problem was that of removing organic, oligomeric and/or polymeric surface fouling from the heterogeneous, precious metal catalyst during the continuous process and of lowering the residual content of methacrolein in the treated catalyst to less than 100 ppm.

An additional problem was that of keeping the catalyst activity as constant as possible during the operative reaction phase, and of effectively counteracting a fall in catalyst activity by suitable measures. A problem associated with this was that of keeping the specific catalyst performance. expressed as mol MMA per kg or litre of catalyst, as constant as possible and of counteracting a fall in this specific catalyst performance and space-time yield by suitable measures.

Furthermore, an additional problem was that of purifying the catalyst, without having to interrupt the process per se, such that said catalyst can be recycled to the reaction or optionally discharged from the reaction, without there being a risk of an exothermic reaction outside of the process.

An additional problem was that of regenerating the catalyst and purifying the removed catalyst for regeneration and workup of the metal constituents contained therein, and also the value-added recovery thereof under the aspect of safe handling of the removed pyrogenic catalyst.

Another problem was that of removing the reaction solution from the catalyst such that said reaction solution does not pose any process risk during the catalyst workup and optionally for the reaction solution to be recycled to the continuous process, such that the overall yield of the process can be designed to be as high as possible.

Another problem was that of also being able to remove disruptive catalyst fines efficiently, optionally during the continuous operation of the process.

Further problems which are not stated explicitly may become apparent from the description, the claims, the examples or the overall context of the present invention.

### Solution

The problems are solved by providing a novel, modified continuous process for the oxidative esterification of aldehydes. This continuous process serves for the production of alkyl methacrylates, the alkyl methacrylates particularly being obtained by oxidative esterification of methacrolein with oxygen and an alcohol in the presence of a heterogeneous catalyst. The heterogeneous catalyst used for this purpose has an oxidic support and at least one precious metal.

In particular, the process according to the invention has the following process steps:
a. removing at least a portion of the catalyst in the form of a suspension, from the reactor,
b. separating the catalyst from the suspension removed in step a.,
c. optionally washing the catalyst from process step b. once or multiple times,
d. thermally treating the catalyst and/or treating the catalyst with a basic solution,
e. adding fresh catalyst to the reactor, and/or
f. adding the reactivated catalyst from process step d. and optionally from c. to the reactor.

The addition of catalyst to the reactor in steps e. and/or f. is essential according to the invention, but it is open as to which of the two fractions, or a combination of both fractions, is added. The particular aspect of the present invention can in particular be seen in process step d., in which methacrolein is finally removed from the removed catalyst in a highly efficient manner. Surprisingly, it has proven possible, by means of this process step d. and optionally c., to remove the inherently toxic, volatile and highly flammable methacrolein particularly efficiently from the removed catalyst.

This includes not only monomeric methacrolein but also oligomers or polymers formed from or with methacrolein. The catalyst removed in this way can then in process step e.—optionally in a further purified state-be returned to the reactor, or else the removed catalyst treated in this way can be safely stored, transported and processed to recover the precious metal. Such an approach, recycling the treated catalyst into the reaction, may be particularly expedient upon the first removal, when the catalyst is still relatively fresh. However, after long run times, it is more expedient to add fresh catalyst according to process step f. and to work up the removed catalyst after the treatment in process step d. such that the precious metal is recovered and for example used to produce new catalyst batches. Mixed forms are also conceivable, e.g. dividing up the removed catalyst according to particle size and recycling the larger catalyst particles-generally together with fresh catalyst-into the reactor, and workup of the smaller particles to recover precious metals, generally gold, platinum or palladium. This approach can also be carried out over the reactor on-stream time as a mid-way phase between a pure approach according to process step e. and an approach according to process step f.

In summary, for a catalyst that is not recycled according to process step f., such a process would be as follows: after the removal in process step a., the separation in process step b, the optional washing in process step c. and the treatment in process step d., the catalyst is treated such that the precious metal is removed from the support material of the catalyst and can be used to produce fresh catalyst. Optionally, the precious metal is removed from the catalyst, obtained in elemental metal form and credited to the client's precious metal account and remunerated.

Preferably, the alcohol is methanol and the alkyl methacrylate is MMA.

In particular, the oxidative esterification can take place at a temperature of between 20 and 120° C., a pH of between 5.5 and 9 and a pressure of between 1 and 20 bar. The reaction is preferably conducted in such a way that the reaction solution contains between 2 and 10 wt % of water.

With regard to the reactor, there are in particular two embodiments:

In the first embodiment, the reactor is a slurry reactor. Here, the catalyst has a geometric equivalent diameter of between 10 and 250 μm, and the removal from the reactor takes place semi-continuously or continuously, particularly preferably by means of sedimentation in an inclined settler. Alternatively, the removal can also take place in batches via an immersed tube or semi-continuously in a circulation stream via a filter candle which may be backwashable.

It has proven particularly favourable for the removal from the reactor to take place by means of sedimentation in an inclined settler, the removal being possible at both outlets of the inclined settler while maintaining the flow and velocity profile of the inclined settler which is present in normal operation without removal of catalyst. As a result, firstly the filtration efficiency of the inclined settler is not disrupted and secondly the penetration of gas bubbles into the inclined settler and the catalyst workup is prevented. When using a lamella separator or inclined settler as a retaining system for the suspension catalyst, it should be taken into consideration that the lower outflow of the apparatus is in principle provided for recycling the degassed two-phase catalyst mixture to the reaction matrix. However, the reaction solution that is continuously guided, outside the inclined settler apparatus, from the reactor to the workup, contains small amounts of catalyst constituents and particles which are optionally filtered via a further stationary filtration unit.

The applicability of the invention is therefore substantially not subject to any limitations with regard to the level of decline of the catalyst slurry: it is possible equally for a catalyst suspension having a particle concentration of up to 20 wt % of catalyst, and for a reaction product matrix containing a proportion of catalyst of considerably less than 1 wt %, to be supplied to the separation and treatment or regeneration depending on the efficiency of the inclined settler and the sedimentation action thereof.

When using an immersed tube, preference is given to placing the immersed tube be placed such that no gas bubbles enter the immersed tube and that at the same time the full grain spectrum of the catalyst is removed.

Ultimately, the catalyst suspension can also be removed directly from the reactor, operated under pressure, which is simple to effect provided that the receiving apparatus is operated at a lower pressure. In this approach, the removal takes place by means of gravity or via different pressure ratios in the discharging and receiving apparatuses, or via a combination of both of these principles. Preferably, the removal of the catalyst slurry and the simultaneous filtration of a reaction-moist particle mass take place in a filtration unit. A reaction-moist particle mass essentially denotes the particulate catalyst that has largely been removed from the reaction medium by filtration but that still contains constituents of organic and inorganic components of the reaction medium. According to the problem of the invention, these components, particularly the methanol and methacrolein fractions, should be considered as highly critical to the further treatment and regeneration because of their toxic properties and particularly because of the fact that, in the presence of air, such organically loaded particle masses can tend to self-ignite or can exhibit adiabatic strong and progressive, even uncontrolled, heat development upon removal and treatment.

In the second embodiment, the reactor is a fixed-bed reactor. When using such a fixed-bed reaction, it has proven advantageous if the catalyst has a geometric equivalent diameter of between 250 μm and 10 mm and the removal from the reactor takes place by means of a discharge or a plurality of discharges from individual fixed-bed units.

As oxidic support, the catalyst generally comprises at least one or more oxides of silicon, aluminium, one or more alkaline-earth metals, and oxides of titanium, zirconium, hafnium, vanadium, niobium, tantalum, yttrium and/or lanthanum.

The precious metal is generally gold, platinum or palladium, but other precious metals, e.g. ruthenium or silver, can also feasibly exhibit catalytic activity. The precious metals are usually present on the surface or in the accessible pore structure of the catalyst particle, of the usually porous support, as particles having a diameter of between 2 and 10 nm.

Furthermore, however, the catalyst may optionally and simultaneously but preferably have an additional metal and/or metal oxide, in particular lead, iron, nickel, zinc and/or cobalt oxide on the surface of the support. In this case, the molar ratio of lead, iron, nickel, zinc and/or cobalt to the precious metal is particularly preferably between 0.1 and 20.

The individual steps of the process can in principle be carried out independently of one another continuously, semi-continuously and/or in a batchwise process. Preference is given to an embodiment of the present invention in which the removal of the catalyst from the reactor in process step a. takes place continuously or semi-continuously, the purification in process steps b. to d. takes place in a batchwise manner, and the recycling of the catalyst or the addition of fresh catalyst in process steps e. and/or f. takes place in a batchwise manner, or in particular semi-continuously. In this context, semi-continuously means that the step sometimes takes place continuously but with relatively long and/or regular interruptions. Conversely, continuous describes carrying out a step without any appreciable interruptions.

The following paragraphs describe in detail various embodiments of the individual process steps:

Process step a. is preferably particularly characterized in that the catalyst is at least partially removed from the reactor during the continuous reaction, preferably in suspended form. In this case, the removed suspension generally contains at least one alkyl methacrylate and methacrolein. Alternatively, it is however also possible according to the invention to stop the reaction and to work up the entire catalyst for the workup according to the above-described process steps before it is returned to the reactor.

In process step b., the separation of the catalyst takes place in the form of a filtration and/or centrifugation, with it also being possible for more than one separation step to be carried out one after another. Before rinsing and washing, the particulate catalyst separated from the reaction solution contains organic constituents from the reaction solution, in particular methanol, water, methacrolein, MMA and salts of methacrylic acid.

In a preferably carried out additional process step c., the catalyst from process step b. is washed with at least one organic solvent and optionally subsequently with water. Process step c. serves for removing critical substances such as methanol and methacrolein in order to ensure that, when removing and handling the deactivated catalyst, removed later, no contact can take place by the release of these substances. A further aim of washing is to remove oxidizable components, since otherwise when the moist material is removed and comes into contact with air, it may ignite. It has been found that while running through a plurality of washing cycles, for example with methanol, is in principle possible and the catalyst becomes depleted in methacrolein, a plurality of passes are however necessary in order to achieve a non-critical MAL concentration. Methacrolein contents of considerably less than 1 wt %, preferably less than 0.1 wt % and particularly preferably less than 100 ppm are considered to be non-critical concentrations

Particularly preferably, in the purification, the first step in the succession is washing with at least one organic solvent. Thereafter, at least one second rinse with the same or with another solvent or solvent mixture can take place. Thereafter, or alternatively as a second purification step, washing with water or an aqueous solution can be carried out.

Preferably, the organic solvent used for the washing is a solvent which is miscible with each of the components of the reaction mixture in any ratio and, at the same time is also particularly capable of dissolving process-related organic salts at more than 1 g salt/l solvent.

Solvents which are mixtures consisting of at least 95 wt % of an alcohol, particularly preferably methanol, and/or acetone, have proven particularly preferable for a first wash with organic solvent. Alternatively preferably, it is also possible to use pure alcohol, in particular methanol and/or acetone.

Very particularly preferably, the organic solvents, in particular for a second wash with organic solvents, are mixtures containing at least 80 wt % diethyl ether, pentane, hexane, cyclohexane, toluene and/or a saturated alkyl ester based on a C1-C8 acid, and optionally at least one of the components comprises alcohol, particularly preferably methanol, acetone and/or MMA.

Particularly preferably, process step c. involves washing twice with organic solvents and subsequently washing at least once with water, the proportion of methacrolein in the catalyst from process step b. being reduced by at least 90 wt % in process step c.

In a particular variant of the process according to the invention, it is possible to recycle at least a portion of the organic solvent used in process step c. into the reaction section or workup section of the process. These may in particular be the plant sections where alcohol, in particular methanol, is present. This may for example be the reactor, or one of the downstream workup columns.

The washing or rinsing of the removed catalyst is generally performed in a closed apparatus in which the catalyst forms or partially thickens a filtercake and has the washing liquid flow through it. In this case, the washing can take place using a backwashable filter housing or a Nutsche filter. It has proven particularly expedient to resuspend the catalyst between the individual washing steps in the washing medium in question. This results in higher washing efficiency or a lower consumption of washing liquid. In addition, the filtration resistance increases due to less compaction of the filtercake, which accelerates the filtration rate. The actual filtration can take place gravimetrically or using pressure, it being possible for the pressure to be applied hydraulically or pneumatically. Particularly preferably, the filtration takes place with the application of an inert gas, for example nitrogen, in order to prevent the formation of an explosive mixture and to accelerate the separation of the liquid. A pressure Nutsche filter or a rotary pressure Nutsche filter have proven to be particularly preferred apparatus for washing the catalyst.

The gravimetric ratio of the respective washing liquid to catalyst is between 1:1 and 100:1, preferably between 1:1 and 10:1, and very particularly preferably between 2:1 and 5:1.

The times of the individual washing steps for the catalyst are not subject to any limitations, however they are typically in the range from 1 minute to 10 hours, with shorter washing times leading to displacement-based washing without any diffusion-based effect in the catalyst pores, also resulting in an increased requirement for washing liquid. Preferably, the time per washing step is between 2 minutes and 1 hour, and very preferably between 5 and 30 minutes. This also applies to the optional treatment with the basic aqueous solution in process step d.

In order to reduce the amount of methacrolein to a minimum, preferably to entirely remove the methacrolein, in process step d. there preferably follows either rinsing with a basic aqueous solution, particularly preferably with an aqueous hydroxide solution, optionally followed by a further washing with an organic solvent, or a thermal treatment of the catalyst. This thermal treatment preferably takes place at a temperature of between 250 and 750° C., particularly preferably of between 300 and 650° C. Alternatively to these two alternatives, but less preferred since it is not necessarily required, it is also possible to combine the two alternatives together.

The basic aqueous solution is for example a solution of an organic or inorganic alkali metal or alkaline-earth metal salt, for example sodium carbonate, sodium hydrogencarbonate, potassium carbonate, potassium hydrogencarbonate, magnesium carbonate, calcium carbonate, sodium hydroxide, potassium hydroxide, magnesium hydroxide, calcium hydroxide or the oxides of sodium, potassium, magnesium or calcium. Very particularly preferably, the basic aqueous solution is a hydroxide solution, very particularly preferably aqueous sodium hydroxide solution.

Particularly preferably, in process step d., use is made of a hydroxide solution which has a pH greater that the pH of the reaction medium in the reactor, preferably has a pH of between 7.5 and 13, and this medium contains dissolved alkali metal and/or alkaline-earth metal hydroxides and water.

The basic aqueous solution may be present at any desired concentration depending on the solubility of the bases. In order to minimize reactions of bases with the oxidic catalyst support, such as the swelling of silica supports, what is referred to as “concrete cancer”, the concentration of the base in the case of a hydroxide is particularly preferably in the range from 0.1 to 25 wt %, in particular preferably between 0.5 and 10 wt % and very particularly preferably between 1 and 5 wt %.

In order to determine the residual content of methacrolein or general organic constituents, the treated catalyst can be resuspended in the form of a sample in an organic solvent, with preference being given here to methanol or chlorinated solvents, and the solution can be examined by means of GC or HPLC for methacrolein or other organic constituents. A direct measurement of the methacrolein content by means of GC headspace analysis over the solid is technically demanding, since rapid polymerization occurs and causes a falsely low value to be determined.

In order to check whether, after the treatment of the catalyst, organic deposits such as absorbates or adsorbates are still present on the surface of the catalyst or in the catalyst pores, a catalyst sample can be examined by means of IR for characteristic wavenumbers in comparison to reference substances. Examination by means of thermogravimetric analysis (TGA) is more technically demanding, with the vaporized compounds or fragments thereof being verified by means of coupling to a mass spectrometer. Comparison to reference substances is also recommended here

The washing of the catalyst in process step c., and the optional process step d., yields a plurality of washing filtrates which contain other materials of value in addition to the oligomeric or polymeric compounds. These washing filtrates may, for example, be the following:
1) reaction mixture
2) organic constituents in the first washing filtrate
3) organic constituents in the second washing filtrate
4) aqueous washing filtrate
5) basic aqueous washing filtrate from process step d.

Optionally, in order to prevent the loss of materials of value, some of the various fractions can be fed to the MMA workup process, preferably after the reactor section of the process, and the materials of value, namely principally methanol, MMA and methacrolein, can be recovered. In this case, the use of methanol alone or in a mixture with MMA, which at the same time constitutes a raw material in MMA synthesis, is naturally particularly advantageous, in particular for this MMA production process.

The following description of an exemplary embodiment serves to clarify the process without limiting the invention in any way.

In a preferred embodiment, the reactor is connected to a first distillation column in which unreacted methacrolein is removed from the reaction mixture in the form of the methacrolein-methanol azeotrope and is returned to the reactor. The bottoms product of this first distillation is subsequently acidified with aqueous sulfuric acid to a pH of less than or equal to 3, in order to convert sodium methacrylate to free methacrylic acid and to hydrolyze disruptive by-products such as methacrolein-methanol acetal. At the same time, the organic sodium salts are converted to inorganic sodium sulfate which, depending on the water and methanol content of the resulting mixture, is present in dissolved form

After addition of water or aqueous acid, the homogeneous substance mixture separates into two phases. In this advantageous embodiment of the process, the organic phase is fed into an extraction column, at the top of which a crude MMA is obtained for further purification. The aqueous phase and the bottoms of the extraction are then fed into a second distillation column, in which, inter alia, methanol and MMA are recovered as tops product. The bottoms product from the second distillation is discharged from the process as wastewater and suitably worked up. The workup is preferably a neutralization, followed by biodegradation of the remaining organics, such that process wastewater is obtained which meets the requirements for municipal wastewater. Other workup or disposal options for the wastewater, with or without neutralization, include extensive evaporation of the volatile constituents, primarily water, for example in a spray-drying step or other suitable processes This makes it possible to isolate sodium sulfate in a relatively pure form and furthermore to at least partially recycle the evaporated quantities of organic substances and water to the process. Because, with a view to the composition, the wastewater can be classified as non-critical, an injection method referred to as deep well injection, in ground formations provided for this purpose, can also be considered.

In the catalyst washing, the reaction mixture is preferably fed into the first distillation column such that the methacrolein contained therein can be recycled to the reaction after distillation. The first organic washing filtrate can also be fed into the first distillation column or optionally into the phase separation before the extraction column. The filtrates starting from the aqueous NaOH solution can be fed into the second distillation column. An alternative position in order to recycle the organic washing solutions with the aim of recovering the material of value in the workup process is the low boilers column which follows the extraction in the downstream section of the workup process.

In process step e., it is optionally possible, but at the same time preferable, for fresh catalyst to be added, particularly preferably in the form of a suspension, e.g. containing water, the alcohol and/or the alkyl methacrylate.

In process step f., finally, organics-moist, purified catalyst is then generally recycled to the reactor. Preferably, this purified catalyst, before being added to the reactor, is suspended in a liquid, preferably containing water, the alcohol and/or the alkyl methacrylate. The addition can take place together with, or separately from, the optional process step e.

For the less preferred alternatives of the process, in which the entire catalyst was removed and the continuous reaction in the reactor was interrupted, the addition as a suspension or directly as a solid can take place, with spraying-for example using cleaning-in-process nozzles-with water being recommended to reduce dust formation or adhesion.

With regard to recycling the treated catalyst to the reactor in process step f. or the addition of fresh catalyst in process step e., it is particularly advantageous if the catalyst to be recycled is added to a circulation stream of the reactor through which reaction solution flows, the influence of this recycling stream on the hydrodynamics of the reactor being kept minimal. To this end, this can for example be converted to a slurry beforehand in the washing and filtration apparatus using reaction solution or a reactant and/or product composition, and pumped or conveyed into a separate container, from which the regenerated catalyst is recycled back to the reactor in a time-delayed manner as a batch, continuously or preferably semi-continuously. The washing and filtration apparatus is preferably flushed again with the reaction solution or a reactant and/or product composition in order to minimize catalyst adhesions. In the case where the oxidative esterification is a process for producing MMA, it is particularly advantageous if, before being returned to the reactor, the catalyst is converted to a slurry with reaction solution, methanol, methacrolein and/or mixtures thereof and is returned with the liquid.

Optionally, in order to prevent or minimize the formation of oligomers or polymers when the catalyst is resuspended and recycled, it is recommended to add a stabilizer, which is preferably the same stabilizer as is also used in the reaction.

It has proven particularly preferable for the separate container to be equipped with internal circulation means or a stirrer, such that the catalyst suspension contained therein can be recycled to the reactor in a homogenized form in terms of solids distribution. Optionally, the container can also be provided with fresh catalyst via an inertizable port, such that a catalyst makeup can additionally take place in parallel to the catalyst regeneration.

Further steps for purifying the removed catalyst can optionally additionally be carried out without having to be explicitly mentioned here. Generally, however, preference is given to no such further steps or to the step described below.

In an additional process step in the purification, in addition to the removal of organic, oligomeric or polymeric impurities from the catalyst surface, an additional step for removing disruptive fines fractions can also take place. This may be advantageous above all if the reaction is carried out in a slurry reactor as described above. To this end, the removed catalyst can be filtered during the purification in order to separate fines fractions having a diameter of less than 10 μm. Depending on its nature, the separated fines fraction is not, or not completely in relation to the entire fines fraction, recycled back into the reactor. It is easiest to initially carry out this method step in a batchwise manner, but continuous or semi-continuous filtrations are also conceivable, although such configurations are technically demanding since the filtered-off solid is recycled. Such a separation can take place for example by centrifugation, preliminary classification or by means of a backwashed filtration.

In order to counteract catalyst concentrations or activities in the reactor, which may fluctuate over time due to the process according to the invention, there is a particular embodiment of the present invention. Here, during or directly after process step a., the internal reactor temperature and/or the internal reactor pressure and/or the stirring speed is increased at least once in comparison to the reaction conditions prior to process step a.

Falling catalyst activity can be observed over an operating period of 8000 h. This occurs even if, according to the invention, a portion of the catalyst is removed and purified before being recycled back to the reactor. Furthermore, the decline is even occasionally observed when fresh catalyst is added after very long run times.

In order to additionally counteract this, the reaction temperature can for example be increased once or several times by 0.5 to 10° C. relative to the starting temperature. Alternatively, or even in addition, the pressure in this operating period can also be raised, for example by 0.1 to 10 bar relative to the starting pressure. In practical terms, these activity-enhancing measures mean that, for example, within an operating period of 8000 h, the temperature is raised from an initial 80° C. up to 90° C., and the pressure in the reactor is raised from an initial 5 bar absolute up to 10 bar.

A third possibility for enhancing activity is increasing the stirrer speed in order to increase gas dispersion and ultimately also the dwell time of the gas bubbles in the reaction zone. This change in parameters can be carried out separately or synchronously, as individual measures or as a combination of measures. Customarily, one of these measures or the combination of at least two of these measures is (are) carried out with the aim of the conversion of methacrolein supplied to the reactor being at least 50%, preferably greater than 60% and particularly preferably greater than 65%.

### EXAMPLES

### Reference Support Production

An enamel-lined reactor was initially charged with 434 kg of silica sol (Köstrosol 1530, primary particles 15 nm, 30 wt % of SiO₂ in H₂O), which was cooled down to 10° C. with vigorous stirring. The silica sol dispersion was adjusted to a pH of 2 with 60% nitric acid. This is done initially in order to break up the basic stabilization, e.g. with sodium oxide.

In a second enamelled vessel, a mixture of 81.2 kg of aluminium nitrate nonahydrate, 55.6 kg of magnesium nitrate hexahydrate and 108.9 kg of demineralized water was made up. The mixture cooled down in the course of dissolution while stirring and had a pH just below 2. After complete dissolution, 3.2 kg of 60% nitric acid was added.

Subsequently, the metal solution was added to the silica sol dispersion in a controlled manner over the course of 30 minutes. On completion of addition, the mixture was heated to 50° C. and the resulting dispersion was gelled for 24 hours, with a pH of 1 at the end. The resultant viscosity was below 10 mPas.

The suspension (solids content of approximately 30 wt %) was pumped at a temperature of 50° C. with a feed rate of 20 kg/h into a pilot spray tower having a diameter of approximately 1.8 m and sprayed therein by means of an atomizer disk at 10 000 revolutions per minute, giving a spherical material. The drying gas supplied was adjusted at 180° C. such that the emerging cold drying gas had a temperature of 120° C. The resultant white spherical material had a residual moisture content of 10% by weight. The residual moisture content was determined by drying to constant weight at 105° C.

The spray-dried material was calcined under air in a rotary tube-like continuous apparatus at 650° C., the dwell time being nearly 45 minutes. The angle of inclination was adjusted to approximately 2° and baffle plates were installed in the rotary tube in order to achieve the dwell time. In order to eliminate nitrogen oxides formed, air was added in counter-current to the solids feed, with the amount of air being metered in such that the loss of solids by the offgas was less than 0.5%.

The white, spherical material obtained was classified by means of sieving and sifting, such that the finished support material had a D10 of 36 μm, a D50 of 70 μm and a D90 of 113 μm. The particle size distribution was determined by means of dynamic image evaluation with a HORIBA Camsizer X2.

### Reference Catalyst Production

An enamel tank with a propeller stirrer was initially charged with 167 kg of demineralized water, and 50 kg of the reference support material was added. The steps that follow were conducted under isothermal conditions by means of steam heating of the reactor. Directly thereafter, a solution of 611 g of aluminium nitrate nonahydrate in 10 kg of demineralized water was added. The suspension was heated to 90° C. and then aged for 15 minutes. 2845 g of cobalt nitrate hexahydrate was dissolved in 20 kg of demineralized water and, on conclusion of the ageing, metered in over the course of 10 minutes and reacted with the support material for 30 minutes.

In parallel, 12.4 l of an NaOH solution was prepared such that the ratio of hydroxide ions to auric acid was 4.75. The NaOH solution was added over the course of 10 minutes, in the course of which the suspension darkened in colour.

After addition of the NaOH solution, 1250 g of an auric acid solution (gold content 41%) in 20 kg of demineralized water was diluted and added to the reaction suspension over the course of 10 minutes and stirred for a further 30 minutes.

The reaction suspension was cooled down to 40° C. after the reaction and pumped into a centrifuge with a filter cloth, with recycling of the filtrate until a sufficient filtercake had been built up. The filtercake was washed with demineralized water until the filtrate had a conductivity below 100 μS/cm, followed by dewatering for 30 minutes Thereafter, the filtercake had a residual moisture content of nearly 30 wt %. The filtrates were first pumped through a selective ion exchanger in order to remove residual cobalt, and then the residual gold was absorbed on activated carbon. The recovery rate of the two metals after the reaction was greater than 99.5%. which was determined by ICP analysis.

Directly after conclusion of the dewatering, the filtercake was dried in a paddle dryer at 105° C. down to a residual moisture content of 2%. The drying process in the paddle dryer was conducted discontinuously within 8 hours with addition of a drying gas—nitrogen in this case.

Directly after the drying, the dried material was fed continuously into the rotary tube described for the reference support material, which was operated at 450° C. under air. The dwell time was adjusted to 30 minutes.

The final catalyst had a loading of 0.91 wt % gold, 1.10 wt % cobalt, 2.7 wt % magnesium, a BET of 236 m²/g, a pore volume of 0.38 ml/g and a pore diameter of 4.1 nm.

### Example 1—Removal of Organics From the Catalyst

1 kg of the reference catalyst was suspended in a stirred-tank reactor equipped with an EKATO Combijet, offgas cooler with added stabilizer, baffles and internal filter candles (nominal 15 μm filter mesh), at 80° C. and 5 bar absolute. The suspension density was 10 wt % and the initial suspension liquid consisted of 30 wt % MMA, 5 wt % water, 1 wt % methacrylic acid and 64 wt % methanol The pH was adjusted to 7 before the catalyst was added

Methacrolein and methanol in a molar ratio of 1:4 was supplied to the reactor, such that 10 mol methacrolein was supplied per hour and per kg of catalyst. The pH was kept constant at 7 by addition of an NaOH solution (4.5 wt % NaOH, 5.5 wt % water, 90 wt % MeOH). The dwell time was 3.7 hours. The reaction outflow was periodically analyzed by means of GC. After 4000 h of operation, the conversion had dropped from 75% to approximately 72%, and the selectivity for MMA remained at 94%.

A catalyst sample was drawn off from the reactor and examined using TGA, and exhibited a mass loss of 4.9% at up to 300° C., 2.7% of which was water The remaining amount was identified by means of IR as being a mixture of oligomers of methacrolein, methacrylic acid and sodium methacrylate.

100 g of the catalyst were pumped via the sample line into a laboratory stirred pressure Nutsche filter. Nitrogen was applied to the catalyst suspension in the Nutsche filter and the liquid was filtered off by suction. Thereafter, the catalyst was resuspended with 300 g MeOH for 10 minutes and filtered off by suction. Subsequently, the catalyst was resuspended in 300 g of a 1.5% aqueous NaOH solution for 10 minutes and filtered off by suction. This was followed by an analogous resuspension-suction filtration cycle with 300 g MeOH and then with 300 g water. Finally, nitrogen was flowed through the catalyst for 10 minutes.

GC analysis showed the following methacrolein levels in the Nutsche filtrates:
Reaction mixture: 8 wt % methacrolein
1st MeOH filtrate: 250 ppm
Aqueous NaOH: 5 ppm
2nd MeOH filtrate: <5 ppm
Water filtrate: <5 ppm

5 g of the treated catalyst were suspended in MeOH (10% solids content) for 14 days and the MeOH was periodically examined by GC. No methacrolein could be detected. Therefore, it can be assumed that no methacrolein escapes from the pores even when the consumed catalyst is being transported.

The treated catalyst was dried overnight at 105° C. and examined by IR and TGA and showed no presence of methacrolein, methyl methacrylate, methacrylic acid or sodium methacrylate or the oligomers thereof.

### Example 2—Testing the Treated Catalyst

20 g of the treated catalyst from Example 1 were loaded into a small reactor set up analogous to the described set up, and the reaction was started. The methacrolein conversion was 74.6% and after 1000 h of operation the same development in catalyst performance was observed as for fresh catalyst. After 1000 h operation was interrupted.

### Example 3—Testing the Treated Catalyst After Calcination

A procedure analogous to Example 2 was performed, except the catalyst was calcined for 5 h at 500° C. before start-up. The methacrolein conversion was 74.9% and after 1000 h of operation the same development in catalyst performance was observed as for fresh catalyst. After 1000 h operation was interrupted.

### Example 4—Continuous or Semi-Continuous Workup of the Catalyst With Continuous Reaction

The reaction system from Example 1 was started with 1 kg of fresh catalyst, and every 250 h 100 g of catalyst were removed from the reactor and treated according to Example 1, with the last step of washing with water being omitted. The catalyst treated in this way was transferred to a separate pressure vessel with a stirrer. In this separate pressure vessel, the catalyst was resuspended in the reaction mixture (10% solids content) and pumped back into the bottom third of the reactor. For every washing procedure, a 5 g sample of catalyst was removed after treatment and 5 g of fresh catalyst were added. Over the operating period of 4000 h, the conversion fell from 75% to 74.7%, corresponding to an improved catalyst on-stream time. The selectivity for MMA was unchanged.

The washing filtrate was separated into phases and stripped by distillation such that the materials of value, MeOH and MMA, were not lost. For continuous recovery of the materials of value from the washing filtrates, feeding in can take place in a continuous MMA purification, as described in U.S. Pat. No. 98,901,05.

Upon IR analysis, the catalyst samples, taken off and dried overnight at 105° C., did not show any traces of methacrolein, methyl methacrylate, methacrylic acid or sodium methacrylate or the corresponding oligomers

### Example 5—Continuous or Semi-Continuous Workup Of The Catalyst With Continuous Reaction and Adaptation of Process Parameters

A procedure analogous to Example 4 was performed, except that, after every 2nd catalyst removal and regeneration, the temperature in the reactor was raised by 0.5° C. and the pressure was raised by 0.25 bar. In total, within 4000 h, the temperature was raised by 4° C. to 84° C. and the pressure was raised by 2 bar to 7 bar. Over the operating period of 4000 h, the conversion fell from 75% to 74.9%, achieving virtually constant catalyst performance. The selectivity for MMA was unchanged.

### Comparative Example 1—Displacement Washing With MeOH and Catalyst Testing

The spent catalyst from Example 1 was washed just once with MeOH. Subsequently, 5 g of catalyst were suspended in MeOH for 14 days (10% solids content). GC analysis showed more than 50 ppm methacrolein in the MeOH within a few hours. When transporting a catalyst treated in this way, it should therefore be assumed that, when the container is open, the methacrolein content in the atmosphere is over 20 ppm (20 mg/m³ air) and therefore over the limit value (TA Luft, Chapter 5.2.5 Organic Substances, Class I).

IR analysis of the catalyst also showed the presence of methacrolein, methacrylic acid and sodium methacrylate or the oligomers thereof.

Testing of the catalyst analogously to Example 2 showed a methacrolein conversion of 72.1% and therefore no improvement in relation to the catalyst prior to treatment.

### Comparative Example 2—Filtration Without Washing

The spent catalyst from Example 1 was flushed as a suspension in reaction mixture onto a pleated filter under a fume hood and pre-dried in air. After 12 hours, the catalyst was dried with the filter paper at 105° C., upon which the filter paper ignited. The catalyst contaminated with ash was disposed of. In a production environment, therefore, there is a high safety risk with insufficient washing.

1. A continuous process for the production of alkyl methacrylates, the alkyl methacrylates being obtained by oxidative esterification of methacrolein with oxygen and an alcohol in the presence of a heterogeneous catalyst having an oxidic support and at least one precious metal, wherein the process comprises:
a. removing at least a portion of the catalyst, or the entire quantity of catalyst, in the form of a suspension, from a reactor,
b. separating the catalyst from the suspension removed in step a.,
c. optionally washing the catalyst from b. once or multiple times,
d. thermally treating the catalyst and/or treating the catalyst with a basic solution,
e. adding a fresh catalyst to the reactor, and/or
f. adding the reactivated catalyst from d. and optionally from c. to the reactor.

2. The process according to claim 1, wherein
the suspension removed in step a. contains at least one alkyl methacrylate and methacrolein, in that
the separation of the catalyst takes place in the form of a filtration and/or centrifugation,
the catalyst from b. is washed with at least one organic solvent and optionally subsequently with water,
an aqueous hydroxide solution is used as the basic solution, and/or the thermal treatment takes place at temperatures between 250 and 750° C.,
the fresh catalyst is added in the form of a suspension, and
the catalyst from d., before being added to the reactor, is suspended in a liquid.

3. The process according to claim 1, wherein, during or directly after a., an internal reactor temperature and/or an internal reactor pressure and/or a stirring speed is increased at least once in comparison to the reaction conditions prior to a.

4. The process according to claim 1, wherein c. contains washing at least once with one or more different organic solvents and subsequently washing at least once with water, the proportion of methacrolein in the catalyst from b. being reduced by at least 90 wt %.

5. The process according to claim 4, wherein at least one organic solvent is a mixture containing at least 80 wt % diethyl ether, pentane, hexane, cyclohexane, toluene, or a saturated alkyl ester based on a C1-C8 acid, and optionally at least one of the components comprises alcohol.

6. The process according to claim 4, wherein at least one of the organic solvents is a mixture consisting of at least 95 wt % of an alcohol.

7. The process according to claim 1, characterized in that wherein, in d., the basic solution is a hydroxide solution which has a pH greater than the pH of a reaction medium in the reactor, and wherein this medium contains a dissolved alkali metal and/or alkaline-earth metal, hydroxides, and water.

8. The process according to claim 1, wherein at least a portion of the organic solvent used in c. is recycled into a reaction section or a workup section of the process.

9. The process according to claim 1, wherein the oxidic support comprises at least one or more oxides of silicon, aluminium, one or more alkaline-earth metals, and also oxides of titanium, zirconium, hafnium, vanadium, niobium, tantalum, yttrium, and/or lanthanum.

10. The process according to claim 1, wherein the at least one precious metal is gold, platinum, or palladium, and said precious metal is present as particles, having a diameter of between 1 and 10 nm, attached to the support.

11. The process according to claim 10, wherein the catalyst additionally comprises lead, iron, nickel, zinc and/or cobalt oxide on the surface of the support, wherein a molar ratio of lead, iron, nickel, zinc and/or cobalt to gold, platinum or palladium is between 0.1 and 20.

12. The process according to claim 1, wherein the removal of the catalyst from the reactor in a. takes place continuously or semi-continuously, the purification in b. to d. takes place in a batchwise manner, and the addition or recycling of the catalyst in e. and/or f. takes place semi-continuously.

13. The process according to claim 1, wherein the reactor is a slurry reactor, the catalyst bas a geometric equivalent diameter of between 10 and 250 μm, and the removal from the reactor takes place by sedimentation in an inclined settler.

14. The process according to claim 1, wherein the reactor is a fixed-bed reactor, the catalyst has a geometric equivalent diameter of between 250 μm and 10 mm, and the removal from the reactor takes place by discharge from individual fixed-bed units.

15. The process according to claim 13, wherein, in b. and/or in c., the removed catalyst is filtered to separate out fines having a diameter of less than 10 μm, and in that the separated fines are not completely recycled into the reactor.

16. The process according to claim 1, wherein the alcohol is methanol and the alkyl methacrylate is methyl methacrylate (MMA), and
wherein the oxidative esterification takes place at a temperature of between 20 and 120° C., a pH of between 5.5 and 9 and a pressure of between 1 and 20 bar, the reaction being carried out such that a reaction solution contains between 2 and 10 wt % water.

17. The process according to claim 1, wherein, in f. and optionally in e., the catalyst is added to a circulation stream of the reactor through which the reaction solution flows, said circulation stream containing a lower concentration of catalyst than the reactor.

18. The process according to claim 1, wherein, after the removal in a., the separation in b., the optional washing in c. and the treatment in d., the catalyst is treated such that the at least one precious metal is removed from the support material of the catalyst, and the at least one precious metal is obtained in elemental metal form and is optionally used for producing fresh catalyst.

19. The process according to claim 1, wherein c. contains washing multiple times with one or more different organic solvents and subsequently washing at least once with water, the proportion of methacrolein in the catalyst from b. being reduced by at least 90 wt %.

20. The process according to claim 4, wherein at least one organic solvent is a mixture containing at least 80 wt % diethyl ether, pentane, hexane, cyclohexane, toluene, or a saturated alkyl ester based on a C1-C8 acid, and optionally at least one of the components comprises methanol, acetone and/or MMA.
